(19) United States
(12) Patent Application Publication
(10) Pub. No.: US 2009/0123637 A1
(43) Pub. Date: May 14, 2009

(54) Novel Food Ingredient and Products Containing the Same

(75) Inventors: Jean-Claude Yvin, Saint-Malo (FR); Nathalie Delzenne, Genval (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: LABORATOIRES GOEMAR, Saint Malo (FR)

(21) Appl. No.: 11/914,708

(22) PCT Filed: May 18, 2006
(86) PCT No.: PCT/FR2006/001131

§ 371 Date: May 29, 2008

(30) Foreign Application Priority Data
May 18, 2005 (FR) 0504984
May 19, 2005 (FR) 0505040

(51) Int. Cl. A23L 1/48

(52) U.S. Cl. 426/658

(57) ABSTRACT

The invention relates to the use of laminarine as food ingredient.

An object of the invention is a novel food ingredient.

The invention also relates to products and in particular foods and more generally nutritional products containing it.

Foods, besides their mineral constituent, contain carbohydrates, lipids and protides. These constituents are metabolized in the organism and in particular in the liver.

In this respect it should be recalled that the liver is the most voluminous organ in the human body and has the most complex metabolism with interdependent excretory and defence functions.

The liver has a strong influence on the nutritional state due to its role in the intermediate metabolism of macronutrients, micronutrients and bile salts.

A diseased liver upsets the digestion, the absorption of nutrients and it can be the cause of vitamin and mineral deficiencies as well as proteolytic malnutrition.

Certain liver dysfunctions are involved in the appearance and development of a modern scourge, namely obesity.

The recommendations of nutrition specialists aimed at encouraging consumers to keep to a diet that will protect the liver and therefore help to combat obesity are a long way from being followed, given the constraints which result from them and the temptation exerted by numerous foods which are sought after for their flavour.

Conferring upon these foods properties that will contribute to the protection, in the sense mentioned above, of the hepatic tissue would constitute an important advance in nutritional science.

The aim which the Applicant company set for itself was therefore to provide a solution to this problem which is still growing in importance.

The Applicant has found that laminarine, which is a β-1,3-glucane of marine origin and which is commonly used in particular in agriculture, where advantage is taken of the properties which allow it to act on plant growth and on the mechanisms of protection of the latter against certain pathogenic agents, exhibited, in a completely unexpected and surprising manner, the property of having a protective effect on the hepatic tissue by its action on triglyceridaemia and on the serum concentrations of a certain number of specific markers of a hepatic disease, including in particular alanine aminotransferase.

The invention therefore relates to a laminarine-based food ingredient, as a novel industrial product, in a mixture, in particular, with a mineral or organic filler optionally constituted by calcium carbonate, talc, silica, lithothamnion, maltodextrin or cellulose derivatives.

The food ingredient thus constituted comprises from 60% to 98% by weight, preferably from 75 to 95% by weight of laminarine.

A typical food ingredient can have the following composition:

Laminarine: 85% by weight

Filler: 15% by weight

According to an advantageous embodiment, the invention relates to nutritional and food products or formulations containing laminarine or the abovementioned laminarine-based food ingredient.

The laminarine can therefore be incorporated in food formulations either directly or in the form of said food ingredient.

Its content in the food formulations does not generally exceed 10% by weight and is, preferably, from 2 to 8% by weight relative to the total mass of the food.

Its content in nutritional products, preferably constituted by tablets or gelatin capsules, is generally from 40 to 80% by weight, the complement to 100 being constituted by mineral and/or organic fillers which can be chosen from the group comprising maltodextrins and cellulose derivatives.

The abovementioned uses of laminarine according to the invention are made possible due to its lack of toxicity which was demonstrated by the Applicant company and due to the fact that it is flavourless and does not therefore have a detrimental effect on the taste of the foods in which it is incorporated.

The term laminarine denotes mixtures of polysaccharides of the β-1,3-glucane type extracted from brown algae, the molecular weight of which is approximately 2500 to approximately 6000.

Laminarine is constituted by a main linear chain of 15 to 35 glucopyranose units joined by β-1,3 acetal links, this main chain carrying a low proportion of branches.

The average degree of polymerization is approximately 25.

Laminarine is presented in a soluble form and in an insoluble form.

For the extraction from brown algae, reference can be made in particular to the international patent application WO 03/045414 by the Applicant company, published on 5 Jun. 2003.

The particular laminarine which was used within the framework of the work which has made it possible to achieve the invention, had a molecular weight of 4500, an average degree of polymerization of 25, and was essentially constituted by polysaccharides with degrees of polymerization ranging from 20 to 30.

It was presented in the form of an odourless and flavourless off-white powder.

The food formulations according to the invention can be constituted according to the non-limitative examples which follow.

### EXAMPLE 1

Chocolate powder containing laminarine.

| | |
|---|---|
| Defatted cocoa | 90% |
| Sugar | 2% |
| Vanilla flavouring | 0.2% |
| Laminarine | 7.7% |
| Cooking salt | 0.1% |

### EXAMPLE 2

Cereal powder which can be used for . . . and containing

| | |
|---|---|
| Precooked corn meal | 80% |
| Extra virgin olive oil | 2.4% |
| Laminarine | 2.1% |
| Maltodextrin | 2% |
| Hydrogenated vegetable fat matter | 5% |
| (copra and palm kernel oil) | |
| Spelt flour | 1% |
| Dehydrated tomatoes | 0.47% |
| Onion powder | 0.53% |
| Sunflower oil | 1% |
| Lactose | 5% |
| Paprika extract | 0.5% |

### EXAMPLE 3

Flavoured beverage containing laminarine

| | |
|---|---|
| Laminarine | 5% |
| Sugar | 2% |
| Lime flavouring | 1% |
| Spring water QSP | 100% |

The nutritional products according to the invention are generally presented in the form of tablets or gelatin capsules, two non-limitative examples of which are given hereafter.

### EXAMPLE 4

### Tablets

450 mg tablets containing:

| | | |
|---|---|---|
| 225 mg of laminarine | i.e. | 50% |
| 176 mg of maltodextrins | i.e. | 39.1% |
| 40 mg of tricalcium phosphate | i.e. | 8.9% |
| 9 mg of magnesium stearate | i.e. | 2% |

For the preparation of the tablets, the constituents are sieved, mixed then compressed into bar shapes. The latter are then introduced into a grinder which transforms them to granules of a size suitable for the stage of compression of the final tablet.

The tablets can be marketed in plain form, film-coated or sugar-coated. They can be packaged in a pillbox or in thermoformed blister packs of 15 or 20 units.

It is possible to consume 2 to 4 a day, in 2 administrations during meals; at regular intervals in order to optimize the nutritional benefits.

### EXAMPLE 5

### Gelatin Capsules

363 mg gelatin capsules of vegetable or marine origin containing:

| | | |
|---|---|---|
| 225 mg of laminarine | i.e. | 78.1% |
| 60 mg of microcrystalline cellulose | i.e. | 20.8% |
| 2 mg of silica | i.e. | 0.7% |
| 1 mg of magnesium stearate | i.e. | 0.4% |

These gelatin capsules can be packaged in pillboxes or in thermoformed blister packs of 15 or 20 units.

They can be consumed at a rate of 2 to 4 gelatin capsules a day, in 2 administrations during meals at regular intervals in order to optimize the nutritional benefits.

1. Laminarine-based food ingredient comprising from 60% to 98%, preferably from 75% to 95% by weight of laminarine as well as a mineral and/or organic filler.

2. Food product containing laminarine or the laminarine-based food ingredient according to claim 1, the laminarine content of the food product being 10% by weight maximum and, preferably, from 2 to 8% by weight relative to the total mass of the food product.

3. Nutritional product preferably constituted by tablets or gelatin capsules and containing from 40 to 80% by weight of laminarine, the complement to 100 being constituted by mineral and/or organic fillers.
